(19) United States
(12) Patent Application Publication
(10) Pub. No.: US 2021/0022944 A1
(43) Pub. Date: Jan. 28, 2021

(54) LOWER-BODY EXOSKELETON USING ELECTROMYOGRAPHY FOR DIRECT FORCE AMPLIFICATION

(71) Applicant: Lockheed Martin Corporation, Bethesda, MD (US)

(72) Inventor: Gavin A. Barnes, St. Cloud, FL (US)

(21) Appl. No.: 17/040,869

(22) PCT Filed: Mar. 27, 2019
(86) PCT No.: PCT/US2019/024293

§ 371 Date: Sep. 23, 2020

Related U.S. Application Data
(60) Provisional application No. 62/649,364, filed on Mar. 28, 2018.

(51) Int. Cl. A61H 3/00; B25J 9/00

(52) U.S. Cl. CPC A61H 3/00; B25J 9/0006; A61H 2230/085; A61H 2201/1642; A61H 2003/007; A61H 2201/1652

(57) ABSTRACT

A lower-body exoskeleton using electromyography for direct force amplification is disclosed. The embodiments relate generally to powered exoskeletons and, in particular, to a powered lower-body exoskeleton using electromyography for direct force amplification, where the powered lower-body exoskeleton has no load-bearing interface to receive an external load, and the powered lower-body exoskeleton has no load-bearing ground contact configured to transfer an external load to the ground.

### RELATED APPLICATION

This application claims the benefit of U.S. Provisional Application No. 62/649,364, filed on Mar. 28, 2018, entitled “LOWER-BODY EXOSKELETON USING ELECTROMYOGRAPHY FOR DIRECT FORCE AMPLIFICATION,” the disclosure of which is hereby incorporated herein by reference in its entirety.

### TECHNICAL FIELD

The embodiments relate generally to exoskeletons and, in particular, to a lower-body exoskeleton using electromyography for direct force amplification.

### BACKGROUND

Much analysis has gone into powered exoskeletons that allow an individual to manipulate external loads with less physical exertion than would be possible without the powered exoskeleton. For example, a powered exoskeleton may be used to allow a user to move relatively heavy items from one location to another location, or to carry an item over a distance.

A powered exoskeleton applies forces to one or more links of an exoskeleton structure to reduce the amount of force that an individual would otherwise have to apply. However, some conventional exoskeletons rely on mechanical sensors that detect a force being applied by the individual to determine when and how much force to apply to a link. Since these mechanical sensors still require the individual to exert a force against the link before the exoskeleton applies its own force to the link, these arrangements can lead to fatigue. Some conventional powered exoskeletons also rely on algorithms, such as predictive algorithms, to determine when and how much force to apply to a link, but these predictive algorithms do not sufficiently mimic the way individuals naturally move, resulting in the individual at times counteracting against the movement of the exoskeleton, which can also lead to fatigue.

### SUMMARY

The embodiments relate generally to powered exoskeletons and, in particular, to a powered lower-body exoskeleton using electromyography for direct force amplification, where the powered lower-body exoskeleton has no load-bearing interface to receive an external load, and the powered lower-body exoskeleton has no load-bearing ground contact configured to transfer an external load to the ground.

In one embodiment, a powered lower-body exoskeleton is provided. The powered lower-body exoskeleton includes a first upper support structure configured to be coupled to a region of a user above a first joint of the user, and a first lower support structure configured to be coupled to a region of the user below the joint of the user. The powered lower-body exoskeleton further includes a first actuator fixed with respect to the first upper support structure. The powered lower-body exoskeleton further includes a first exoskeleton link having a first end and a second end, the first end being coupled to the first actuator and the second end being coupled to the first lower support structure, wherein the first actuator is configured to selectively move the first exoskeleton link in response to an actuator command. The powered lower-body exoskeleton further includes a first electromyography (EMG) sensor configured to generate first EMG sensor data based on a muscle contraction of a muscle of the user. The powered lower-body exoskeleton further includes a controller communicatively coupled to the first EMG sensor and to the first actuator. The controller is configured to receive the first EMG sensor data from the first EMG sensor, and communicate a first actuator command that is based on the first EMG sensor data to the first actuator to cause the first actuator to impart an actuator force on the first exoskeleton link to cause the first exoskeleton link to move. The powered lower-body exoskeleton has no load-bearing interface to receive an external load, and the powered lower-body exoskeleton has no load-bearing ground contact configured to transfer an external load to the ground.

Those skilled in the art will appreciate the scope of the disclosure and realize additional aspects thereof after reading the following detailed description of the embodiments in association with the accompanying drawing figures.

### BRIEF DESCRIPTION OF THE DRAWINGS

The accompanying drawing figures incorporated in and forming a part of this specification illustrate several aspects of the disclosure and, together with the description, serve to explain the principles of the disclosure.

FIG. 1 is a block diagram of a system for lower-body exoskeleton control using electromyography (EMG) for direct force amplification, according to an embodiment;

FIGS. 2A-2C illustrate front, side, and back view diagrams, respectively, of a powered lower-body exoskeleton to facilitate human locomotion according to one embodiment;

FIG. 3 is a diagram of the lower-body exoskeleton illustrated in FIGS. 2A-2C, according to one embodiment;

FIG. 4 is a diagram of a lower-body exoskeleton, according to another embodiment;

FIG. 5 is a diagram of a lower-body exoskeleton, according to another embodiment;

FIGS. 6A-6C illustrate front, side, and back view diagrams, respectively, of a powered lower-body exoskeleton to facilitate human locomotion according to another embodiment;

FIGS. 7A-7B illustrate front and side view diagrams, respectively, of a powered lower-body exoskeleton to facilitate human locomotion according to another embodiment; and

FIG. 8 is a block diagram of a computing device, which may comprise or compose the controller illustrated in any of FIGS. 1-7B, according to an embodiment.

### DETAILED DESCRIPTION

The embodiments set forth below represent the information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

Any flowcharts discussed herein are necessarily discussed in some sequence for purposes of illustration, but unless otherwise explicitly indicated, the embodiments are not limited to any particular sequence of steps. The use herein of ordinals in conjunction with an element is solely for distinguishing what might otherwise be similar or identical labels, such as “first exoskeleton link” and “second exoskeleton link,” and does not imply a priority, a type, an importance, or other attribute, unless otherwise stated herein. The term “about” used herein in conjunction with a numeric value means any value that is within a range of ten percent greater than or ten percent less than the numeric value.

As used herein and in the claims, the articles “a” and “an” in reference to an element refers to “one or more” of the element unless otherwise explicitly specified. The word “or” as used herein and in the claims is inclusive unless contextually impossible. As an example, the recitation of A or B means A, or B, or both A and B.

The embodiments relate generally to a powered lower-body exoskeleton used to enhance human locomotion. The powered lower-body exoskeleton reduces fatigue, allowing a user to travel farther while expending less energy than the user would expend without the powered lower-body exoskeleton. The powered lower-body exoskeleton does not rely on mechanical sensors that detect a force being applied by the individual to determine when and how much force to apply to a link because such mechanical sensors require the individual to exert a force against the link before the exoskeleton applies its own force to the link, and thus cause fatigue. The powered lower-body exoskeleton also does not rely on algorithms, such as predictive algorithms, to determine when and how much force to apply to a link, because such algorithms do not sufficiently mimic the way individuals naturally move, resulting in the individual at times counteracting against the movement of the exoskeleton, which also leads to fatigue. Rather, the embodiments rely on electromyographic (EMG) sensors and direct force amplification to generate actuator commands at a speed that causes movement of an exoskeleton link within milliseconds of a contraction of a user's muscles.

In particular, in response to a contraction of a muscle of a human user, an EMG sensor generates EMG sensor data that quantifies the muscle contraction. A controller receives the EMG sensor data, determines an actuator command based on the EMG sensor data, and communicates the actuator command to an actuator associated with an exoskeleton joint.

One advantage of this arrangement is that the exoskeleton joint can be instructed to move in response to the muscle contraction of the user before the muscle causes the user's joint to actually move. Since the delay between the electrochemical reaction, which causes the muscle contraction, and the actual movement of the human user's joint is greater than the time required for the EMG sensor and the controller to generate and utilize the EMG sensor data to cause the actuator to move, the powered exoskeleton can accurately mimic the actual movements of the human user without the need for conventional reactive or predictive systems, which can lead to fatigue.

As used herein, the term “electromyography” or “EMG” refers to the detection and recording of the electrical activity of muscle tissue resulting from electrochemical reactions in the muscle tissue. When EMG is used with skeletal muscle tissue, i.e., muscles operatively connected to one or more skeletal bones (or other types of body segments), EMG can detect contraction of the muscle tissue before the muscle tissue has contracted with sufficient force to actually move the joint.

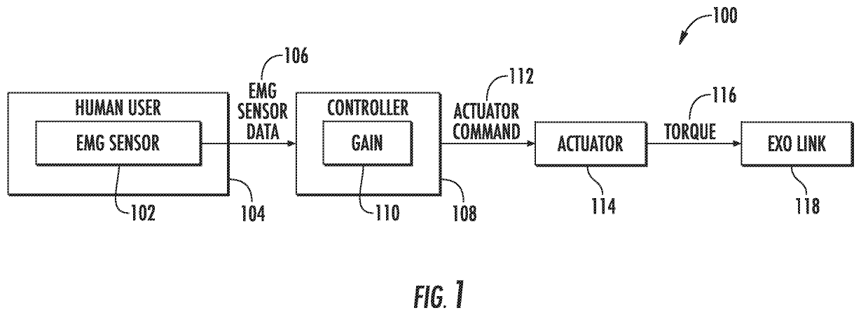

Referring now to FIG. 1, a block diagram of an exoskeleton control system 100 for a powered exoskeleton is illustrated according to an embodiment. The exoskeleton control system 100 includes at least one EMG sensor 102 that engages a human user 104 proximate a muscle of the human user 104. The EMG sensor 102 is configured to quantify a muscle contraction of the muscle of the human user 104. This may be accomplished, for example, by detecting a magnitude of an electrochemical reaction that occurs in the muscle in response to a neurological instruction from the brain of the human user 104 to contract the muscle.

The EMG sensor 102 provides EMG sensor data 106 based on the detected electrochemical reaction to a controller 108, which may be a single device in communication with multiple EMG sensors 102 or may be a plurality of devices each associated with a subset of EMG sensors 102, as desired. In one embodiment, the EMG sensor data 106 is raw EMG sensor data, which may first be high-pass filtered with a second-order Butterworth filter with a cutoff frequency of about 50 Hz to remove motion artifacts. The signal may then be full wave rectified and low-pass filtered using a second-order Butterworth filter with a cutoff frequency of about 4 Hz. The resulting signal may be referred to as a linear envelope.

The controller 108 then determines a gain 110 for inclusion in an actuator command 112 to be sent to an actuator 114. In this embodiment, the actuator command 112 is a function of the gain 110 and the EMG sensor data 106. For example, the gain 110 may be a predetermined value, and multiplied by a magnitude component of the EMG sensor data 106 to derive the actuator command 112. Notably, in this embodiment, predictive mechanisms are not utilized to determine the actuator command 112, and thus, in contrast to systems that utilize predictive mechanisms, the embodiments reduce lag and more closely mimic the natural movements of the human user 104. In response to the actuator command 112, the actuator 114 applies a torque 116 (or other force) containing the gain 110 as a component to one or more exoskeleton links 118. The actuator 114 may be an electromechanical driven actuator mechanism, a cable driven actuator mechanism, or any other suitable actuator mechanism.

Using the EMG sensor data 106 and the gain 110 to produce the actuator command 112 in this manner may be referred to as direct force amplification, because the exertion force produced by the muscle is directly translated into the amplified torque 116 by the exoskeleton control system 100 in real time. It takes about 50 ms from the generation of the neurological instruction for the electrochemical reaction to occur in the muscle, and it takes about 150 ms from the electrochemical reaction for the muscle to actually contract. Since 50 ms is enough time for the exoskeleton control system 100 to detect the electrochemical impulse, generate the actuator command 112, and apply the torque 116 to the exoskeleton link 118, the exoskeleton control system 100 is able to apply the torque 116 to the exoskeleton link 118 associated with an exoskeleton joint faster than the muscle of the human user 104 can contract. This allows the exoskeleton link 118 to “respond” to movement by the human user 104 before the movement occurs. This in turn reduces fatigue in the human user 104 because the exoskeleton link 118 is already in motion by the time the muscle of the human user 104 is able to exert force against the exoskeleton link 118.

In contrast, a conventional reactive control system requires a muscle of a human user to exert a force against an exoskeleton link to trigger movement of the exoskeleton link. Even if the exertion is small, this causes increased fatigue to the human user over time. Similarly, a conventional predictive control system uses an algorithm to predict future movement of the human user based on predetermined movements for the human user, such as a walking gait. However, if the human user makes a movement that is not predicted by the control system, the human user will exert force against the powered exoskeleton, which also causes increased fatigue over time. By using EMG force amplification, however, the exoskeleton control system 100 can accurately mimic and track the movement of the human user 104 in real time, and potentially before the human user 104 actually moves or exerts any force against the exoskeleton link 118. As a result, the exoskeleton control system 100 can be calibrated to apply more gain 110 to the actuator command 112, by adding a calibration value to the gain 110 or multiplying the gain 110 by a calibration value for example, in response to the EMG sensor data 106 indicating an electrochemical reaction having a smaller magnitude. In this manner, more torque 116 can be applied to the exoskeleton link 118 in response to a smaller exertion by the human user 104, and with less exertion by the human user 104 against the exoskeleton link 118 itself. This in turn can allow the human user 104 to carry heavier loads with less fatigue over time.

It should be understood that while the controller 108 in this example is configured to generate the gain 110 to cause the actuator command 112 to be directly proportional, e.g. linearly proportional, to a magnitude component of the EMG sensor data 106, the gain 110 can be generated using additional criteria as well. For example, the controller 108 may be calibrated to take into account a fatigue level of the human user 104. A fatigue level of the human user 104 can be predicted based on the measured EMG sensor data 106 over time, by a predictive algorithm, or by baseline calibration data obtained from the human user 104 via a calibration application. For example, by measuring the EMG sensor data 106 produced by a maximum voluntary contraction of the human user 104's muscle in a controlled environment, an appropriate amount of the gain 110 may be provided so that the human user 104 is not required to reach the maximum voluntary contraction in order to achieve maximum power for the actuator 114. In this manner, by reducing the amount of effort required to produce maximum torque 116 over time, fatigue can be reduced. The calibration value can be based on this maximum voluntary contraction, or on an estimated present voluntary maximum contraction based on a predetermined maximum voluntary contraction of the muscle of the human user 104 and on an estimated fatigue level of the muscle, for example.

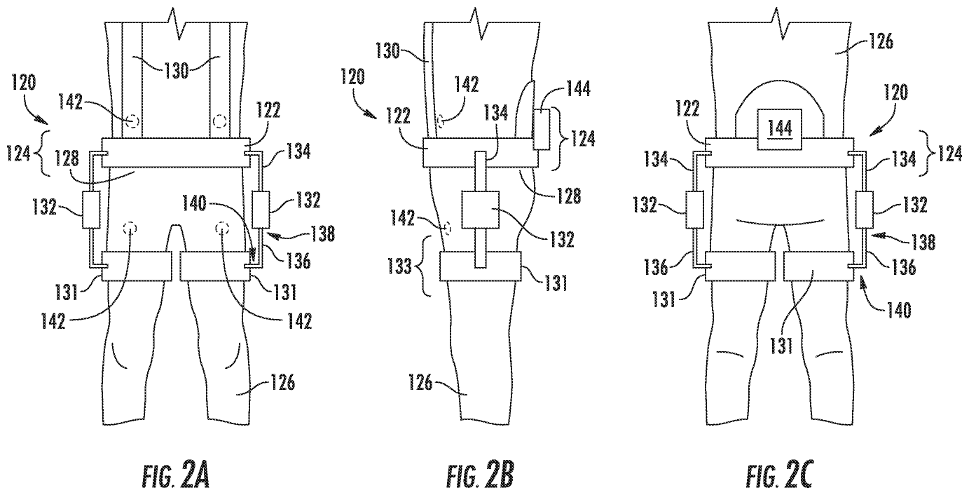

FIGS. 2A-2C illustrate front, side, and back view diagrams, respectively, of a powered lower-body exoskeleton 120 to facilitate human locomotion according to one embodiment. The powered lower-body exoskeleton 120 includes a first upper support structure 122 configured to be coupled to a region 124 of a user 126 above a first joint 128 of the user 126. In this example, the first joint 128 is a hip joint of the user 126, and the region 124 of the user 126 above the first joint 128 of the user 126 is a hip/waist region. The first upper support structure 122 may comprise, for example, a waist belt that is configured to be coupled to the pelvic region of the user 126.

In some embodiments, a suspension mechanism 130, such as suspenders, is configured to be worn over shoulders of the user 126, and the suspension mechanism 130 is configured to couple to the first upper support structure 122. In one embodiment, the suspension mechanism 130 comprises suspenders. The suspension mechanism 130 can bear the weight of the powered lower-body exoskeleton 120, and may also help prevent the lower-body exoskeleton 120 from rotating about the user 126 and maintain proper alignment with the user 126.

The powered lower-body exoskeleton 120 has a first lower support structure 131 configured to be coupled to a region 133 of the user 126 below the first joint 128 of the user 126. In this embodiment, the region 133 of the user 126 below the first joint 128 of the user 126 is a thigh region. A first actuator 132 is fixed with respect to the first upper support structure 122. The first actuator 132 may be fixed directly to the first upper support structure 122, or indirectly via an exoskeleton link 134. A first exoskeleton link 136 has a first end 138 and a second end 140. The first end 138 is coupled, directly, or indirectly, to the first actuator 132, and the second end 140 is coupled to the first lower support structure 131, wherein the first actuator 132 is configured to selectively move the first exoskeleton link 136 in response to an actuator command. The movement may be in a plane that is parallel to a sagittal plane of the user 126 (i.e., a plane that is substantially parallel to the direction of forward movement of the user 126).

A first EMG sensor 142 is coupled to the user 126 and is configured to generate first EMG sensor data based on a muscle contraction of a muscle of the user 126. The first EMG sensor 142 may be coupled, for example, to a thigh muscle of the user 126, or any other muscle of the user 126 that contracts when the user 126 initiates locomotion. In some embodiments, EMG sensor(s) 142 may also be coupled to muscles in or near the pelvic region. In one embodiment, the first EMG sensor 142 comprises an electrode configured to engage the user 126 proximate the respective muscle, detect an electrochemical reaction in the muscle corresponding to the muscle contraction of the muscle, and generate the first EMG sensor data in response to detecting the electrochemical reaction in the muscle. In some embodiments, the electrode is a skin electrode configured to engage a skin surface of the user 126 proximate the muscle. In some embodiments, the electrode is a subcutaneous electrode configured to be disposed under a skin surface of the user 126 proximate the muscle. In some embodiments, the electrode is configured to be inserted into the muscle.

The powered lower-body exoskeleton 120 includes a controller 144 communicatively coupled, wired or wirelessly, to the first EMG sensor 142 and to the first actuator 132. The controller 144 is configured to receive first EMG sensor data from the first EMG sensor 142, and communicate a first actuator command that is based on the first EMG sensor data to the first actuator 132 to cause the first actuator 132 to impart an actuator force on the first exoskeleton link 136 to cause the first exoskeleton link 136 to move. Notably, the powered lower-body exoskeleton 120 has no load-bearing interface to receive an external load, and the powered lower-body exoskeleton 120 has no load-bearing ground contact configured to transfer an external load to the ground.

While for purposes of illustration only the portion of the powered lower-body exoskeleton 120 associated with one leg of the user 126 is illustrated and discussed, it will be apparent that the powered lower-body exoskeleton 120 may contain the same elements discussed herein for both of the legs of the user 126.

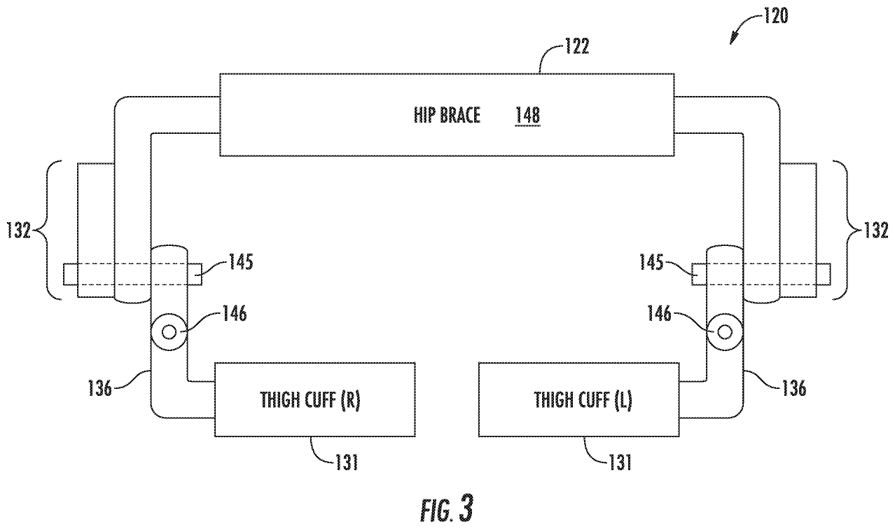

FIG. 3 is a diagram of the powered lower-body exoskeleton 120 according to one embodiment. The powered lower-body exoskeleton 120 includes a pair of actuators 132, one for each leg of the user 126. Each actuator 132 is configured to, in response to an actuator command, impart an actuator force to cause the first exoskeleton link 136 to move. The first exoskeleton link 136 may include a hinge 146. The powered lower-body exoskeleton 120 may include a hip brace 148 that is configured to rest against a lower back region of the user 126. The hip brace 148 may include the lower support structure 122 (FIGS. 2A-2C). Axels 145 are configured to drive respective right and left exoskeleton links 136. Hinges 146 facilitate rotation of the legs of the user 126 about the longitudinal axis of the legs as the user 126 moves.

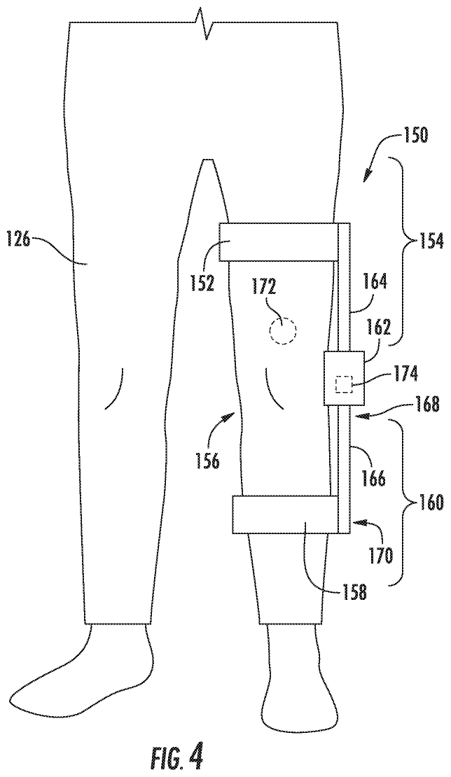

FIG. 4 illustrates a powered lower-body exoskeleton 150 to facilitate human locomotion according to another embodiment. The powered lower-body exoskeleton 150 includes a first upper support structure 152 configured to be coupled to a region 154 of the user 126 above a first joint 156 of the user 126. In this example, the first joint 156 is a knee joint of the user 126, and the region 154 of the user 126 above the first joint 156 of the user 126 is a thigh region. The first upper support structure 152 may comprise, for example, a belt that is configured to be coupled to the thigh region of the user 126.

The powered lower-body exoskeleton 150 has a first lower support structure 158 configured to be coupled to a region 160 of the user 126 below the joint 156 of the user 126. In this embodiment, the region 160 of the user 126 below the first joint 156 of the user 126 is a calf region. A first actuator 162 is fixed with respect to the first upper support structure 152. The first actuator 162 may be fixed directly to the first upper support structure 152, or indirectly via an exoskeleton link 164. A first exoskeleton link 166 has a first end 168 and a second end 170. The first end 168 is coupled, directly, or indirectly, to the first actuator 162, and the second end 170 is coupled to the first lower support structure 158, wherein the first actuator 162 is configured to selectively move the first exoskeleton link 166 in response to an actuator command.

A first EMG sensor 172 is coupled to the user 126 and is configured to generate first EMG sensor data based on a muscle contraction of a muscle of the user 126. The first EMG sensor 172 may be coupled, for example, to a thigh muscle of the user 126, or any other muscle of the user 126 that contracts when the user 126 initiates locomotion. In one embodiment, the first EMG sensor 172 comprises an electrode configured to engage the user 126 proximate the respective muscle, detect an electrochemical reaction in the muscle corresponding to the muscle contraction of the muscle, and generate the first EMG sensor data in response to detecting the electrochemical reaction in the muscle.

The powered lower-body exoskeleton 150 includes a controller 174 communicatively coupled, wired or wirelessly, to the first EMG sensor 172 and to the first actuator 162. The controller 174 is configured to receive the first EMG sensor data from the first EMG sensor 172, and communicate a first actuator command that is based on the first EMG sensor data to the first actuator 162 to cause the first actuator 162 to impart an actuator force on the first exoskeleton link 166 to cause the first exoskeleton link 166 to move. Notably, the powered lower-body exoskeleton 150 has no load-bearing interface to receive an external load, and the powered lower-body exoskeleton 150 has no load-bearing ground contact configured to transfer an external load to the ground. The term “external load” refers to a device or other thing separate from the user 126, such as a tool, a backpack, or the like.

While for purposes of illustration only the portion of the powered lower-body exoskeleton 150 associated with one leg of the user 126 is discussed, it will be appreciated that the powered lower-body exoskeleton 150 contains the same elements discussed herein for both of the legs of the user 126.

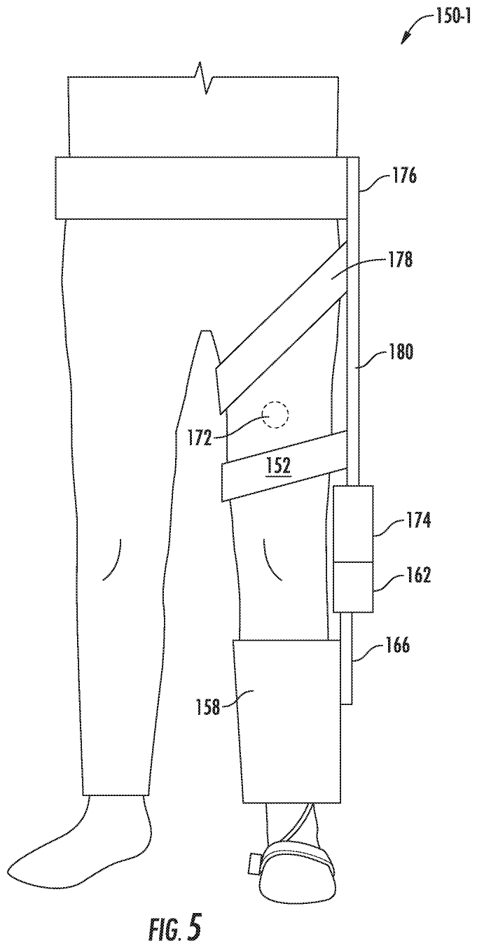

FIG. 5 is a diagram of a lower-body exoskeleton 150-1, according to another embodiment. The lower-body exoskeleton 150-1 is substantially similar to the lower-body exoskeleton 150 discussed above with regard to FIG. 4 except as otherwise discussed herein. In this embodiment, the lower-body exoskeleton 150-1 includes a waist belt 176 coupled to an upper thigh strap 178. A link 180 couples the upper thigh strap 178 to the first upper support structure 152. The waist belt 176, upper thigh strap 178, and link 180 help support the weight of the lower-body exoskeleton 150-1, and help keep the lower-body exoskeleton 150-1 in proper alignment with the user 126 by inhibiting rotation of the lower-body exoskeleton 150-1 about the body of the user 126.

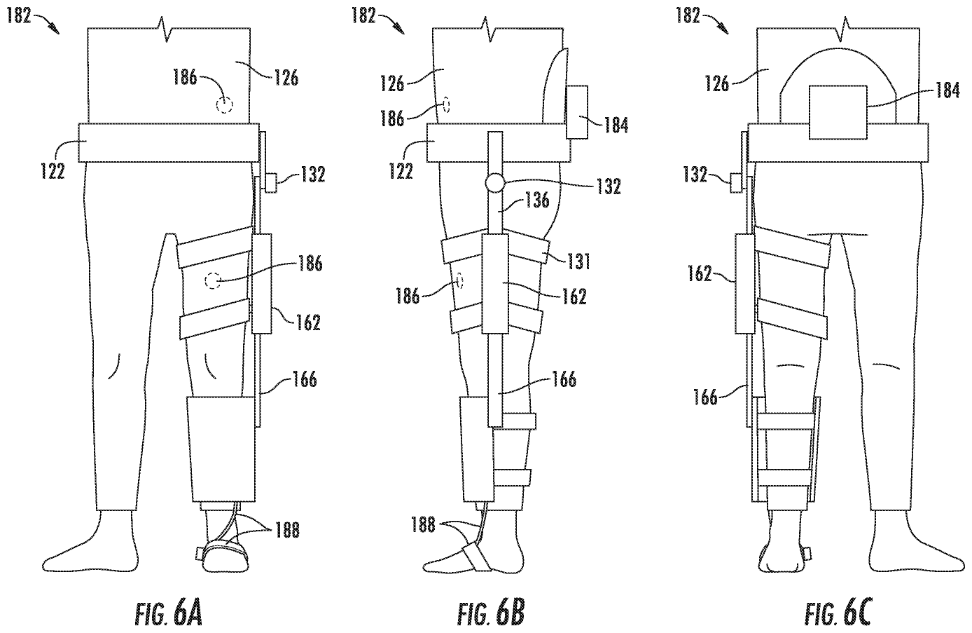

FIGS. 6A-6C illustrate front, side, and back view diagrams, respectively, of a powered lower-body exoskeleton 182. The powered lower-body exoskeleton 182 is essentially a combination of the powered lower-body exoskeletons 120 and 150-1 illustrated above. A controller 184 receives EMG sensor data from a plurality of EMG sensors 186, and sends actuator commands to actuators 132, 162 to facilitate locomotion of the user 126. The powered lower-body exoskeleton 182 may include a harness mechanism 188 that couples to the foot of the user 126 to help prevent rotation of the components about the legs of the user 126. Note that the harness mechanism 188 includes no load-bearing ground contact.

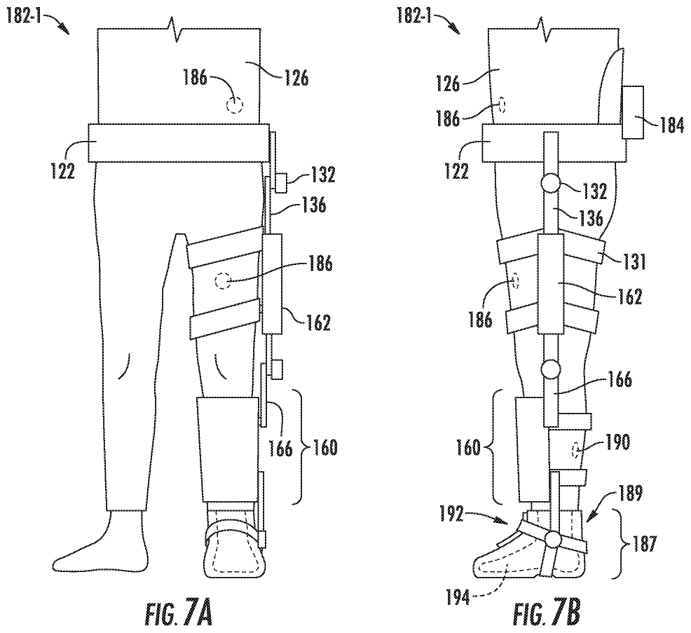

FIGS. 7A-7B illustrate front and side view diagrams, respectively, of a powered lower-body exoskeleton 182-1 to facilitate human locomotion according to another embodiment. The powered lower-body exoskeleton 182-1 is substantially similar to the powered lower-body exoskeleton 182 discussed above with regard to FIGS. 6A-6C except as otherwise discussed herein. In this embodiment, the powered lower-body exoskeleton 182-1 includes an ankle joint mechanism 187 that includes an actuator 189 coupled to the controller 184. An EMG sensor 190 sends EMG sensor data to the controller 184 that quantify muscle contractions in the calf region 160 of the user 126. The ankle joint mechanism 187 includes a harness 192 that is coupled to a foot 194 of the user 126.

By measuring several different muscles associated with different joints, the powered lower-body exoskeletons disclosed herein can perform complex movements in tandem with actual movements of the user 126. Unlike reactive or predictive systems that rely on movements of a user that have already occurred, the measurements received from the EMG sensors can be used to accurately determine movement of an associated joint of the user 126 before the actual movement occurs. In this manner, the powered lower-body exoskeletons disclosed herein can enhance the movement of the user 126 while minimizing resistance by the powered lower-body exoskeletons against the actual movements of the user 126.

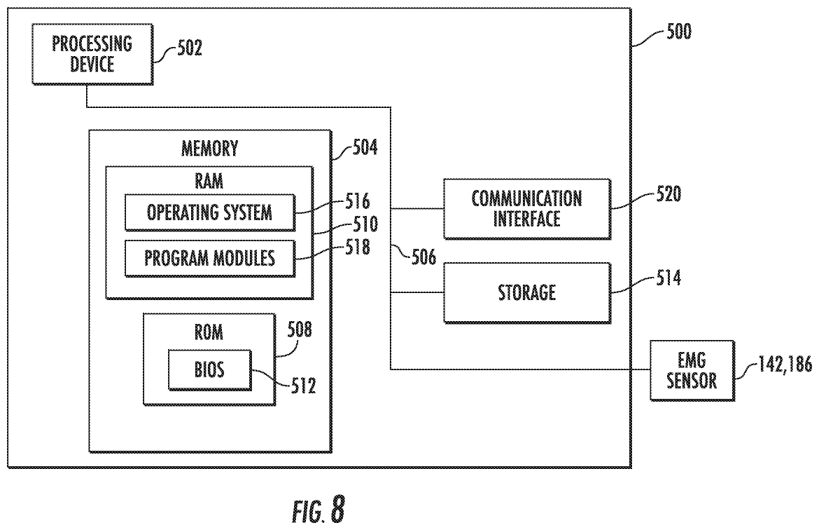

FIG. 8 is a block diagram of a computing device 500, which may comprise or compose the controllers illustrated in any of FIGS. 1-7B, according to an embodiment. The computing device 500 includes a processing device 502, a memory 504, and a system bus 506. The system bus 506 provides an interface for system components including, but not limited to, the memory 504, the processing device 502, one or more EMG sensors 102, etc. The processing device 502 can be any commercially available or proprietary processing device or processing devices.

The system bus 506 may be any of several types of bus structures that may further interconnect to a memory bus (with or without a memory controller), a peripheral bus, and/or a local bus using any of a variety of commercially available bus architectures. The memory 504 may include a volatile memory 510 (e.g., random access memory (RAM)) and/or a non-volatile memory 508 (e.g., read only memory (ROM), erasable programmable read only memory (EPROM), electrically erasable programmable read only memory (EEPROM), etc.). A basic input/output system (BIOS) 512 may be stored in the non-volatile memory 508, and can include the basic routines that help to transfer information between elements within the computing device 500. The volatile memory 510 may also include a high-speed RAM, such as static RAM for caching data.

The computing device 500 may further include or be coupled to a computer-readable storage 514, which may comprise, for example, an internal or external hard disk drive (HDD) (e.g., enhanced integrated drive electronics (EIDE) or serial advanced technology attachment (SATA)), HDD (e.g., EIDE or SATA) for storage, flash memory, or the like. The computer-readable storage 514 and other drives, associated with computer-readable media and computer-usable media, may provide non-volatile storage of data, data structures, computer-executable instructions, and the like. Although the description of computer-readable media above refers to an HDD, it should be appreciated by those skilled in the art that other types of media which are readable by a computer, such as floppy disks, magnetic cassettes, flash memory cards, cartridges, and the like, may also be used in the exemplary operating environment, and further, that any such media may contain computer-executable instructions for performing novel methods of the disclosed architecture.

A number of modules can be stored in the computer-readable storage 514 and in the volatile memory 510, including an operating system 516 and one or more program modules 518, which may implement the functionality described herein in whole or in part. It is to be appreciated that the embodiments can be implemented with various commercially available operating systems 516 or combinations of operating systems 516.

A portion of the embodiments may be implemented as a computer program product stored on a transitory or non-transitory computer-usable or computer-readable storage medium, such as the computer-readable storage 514, which includes complex programming instructions, such as complex computer-readable program code, configured to cause the processing device 502 to carry out the steps described herein. Thus, the computer-readable program code can comprise software instructions for implementing the functionality of the embodiments described herein when executed on the processing device 502. As noted above, the processing device 502, in conjunction with the program modules 518 in the volatile memory 510, may serve as the controller for the exoskeletons disclosed herein, the controller being configured to, or adapted to, implement the functionality described herein. The computing device 500 may also include a communication interface 520, suitable for communicating with a network as appropriate or desired.

Those skilled in the art will recognize improvements and modifications to the preferred embodiments of the disclosure. All such improvements and modifications are considered within the scope of the concepts disclosed herein and the claims that follow.

What is claimed is:

1. A powered lower-body exoskeleton comprising:
a first upper support structure configured to be coupled to a region of a user above a first joint of the user;
a first lower support structure configured to be coupled to a region of the user below the joint of the user;
a first actuator fixed with respect to the first upper support structure;
a first exoskeleton link having a first end and a second end, the first end being coupled to the first actuator and the second end being coupled to the first lower support structure, wherein the first actuator is configured to selectively move the first exoskeleton link in response to an actuator command;
a first electromyography (EMG) sensor configured to generate first EMG sensor data based on a muscle contraction of a muscle of the user; and
a controller communicatively coupled to the first EMG sensor and to the first actuator, the controller configured to:
receive the first EMG sensor data from the first EMG sensor; and
communicate a first actuator command that is based on the first EMG sensor data to the first actuator to cause the first actuator to impart an actuator force on the first exoskeleton link to cause the first exoskeleton link to move;
wherein the powered lower-body exoskeleton has no load-bearing interface to receive an external load, and the powered lower-body exoskeleton has no load-bearing ground contact configured to transfer an external load to the ground.

2. The powered lower-body exoskeleton of claim 1 wherein:
the first joint is a hip joint of the user;
the region of the user above the first joint of the user is a pelvic region;
the region of the user below the first joint of the user is a thigh region; and
the first EMG sensor is configured to be coupled to a muscle in the thigh region of the user.

3. The powered lower-body exoskeleton of claim 2 wherein the first upper support structure comprises a strap configured to be wrapped about a waist of the user, and the first lower support structure comprises a strap configured to be wrapped about a thigh of the user.

4. The powered lower-body exoskeleton of claim 1 wherein:
the first joint is a knee joint of the user;
the region of the user above the first joint of the user is a thigh region;
the region of the user below the first joint of the user is a calf region; and
the first EMG sensor is configured to be coupled to a muscle in the thigh region of the user.

5. The powered lower-body exoskeleton of claim 4 wherein the first upper support structure comprises a strap configured to be wrapped about a thigh of the user, and the first lower support structure comprises a strap configured to be wrapped about a calf of the user.

6. The powered lower-body exoskeleton of claim 1 wherein:
the first joint is a hip joint of the user;
the region of the user above the first joint of the user is a pelvic region;
the region of the user below the first joint of the user is a thigh region; and
the first EMG sensor is configured to be coupled to a muscle in the thigh region of the user, and further comprising:
a second upper support structure configured to be coupled to the thigh region of the user;
a second lower support structure configured to be coupled to a calf region of the user;
a second actuator fixed with respect to the second upper support structure;
a second exoskeleton link having a first end and a second end, the first end of the second exoskeleton link being coupled to the second actuator and the second end of the second exoskeleton link being coupled to the second lower support structure, wherein the second actuator is configured to selectively move the second exoskeleton link in response to an actuator command; and
a second EMG sensor configured to generate second EMG sensor data based on a muscle contraction of a muscle of the user;
wherein the controller is communicatively coupled to the second EMG sensor and to the second actuator, the controller configured to:
receive the second EMG sensor data from the second EMG sensor; and
communicate a second actuator command that is based on the second EMG sensor data to the second actuator to cause the second actuator to impart an actuator force on the second exoskeleton link to cause the second exoskeleton link to move.

7. The powered lower-body exoskeleton of claim 6 further comprising a harness mechanism configured to be coupled to a footwear of the user, the harness mechanism configured to inhibit movement of the second exoskeleton link in a downward direction, and to inhibit rotation of the second exoskeleton link about a calf of the user.

8. The powered lower-body exoskeleton of claim 7 wherein the harness mechanism has no rigid structure that contacts the ground.

9. The powered lower-body exoskeleton of claim 1, wherein the first EMG sensor comprises an electrode configured to:
engage the user proximate the muscle;
detect an electrochemical reaction in the muscle corresponding to the muscle contraction of the muscle; and
generate the first EMG sensor data in response to detecting the electrochemical reaction in the muscle.

10. The powered lower-body exoskeleton of claim 9, wherein the electrode is a skin electrode configured to engage a skin surface of the user proximate the muscle.

11. The powered lower-body exoskeleton of claim 9, wherein the electrode is a subcutaneous electrode configured to be disposed under a skin surface of the user proximate the muscle.

12. The powered lower-body exoskeleton of claim 9, wherein the electrode is further configured to be inserted into the muscle.

13. The powered lower-body exoskeleton of claim 1, wherein the actuator command comprises a gain component, wherein the actuator command is directly proportional to a magnitude component of the first EMG sensor data by a factor of the gain component.

14. The powered lower-body exoskeleton of claim 13, wherein the gain component is at least partially based on a calibration value.

15. The powered lower-body exoskeleton of claim 14, wherein the calibration value is based on a predetermined maximum voluntary contraction of the muscle of the user.

16. The powered lower-body exoskeleton of claim 14, wherein the calibration value is based on an estimated present maximum voluntary contraction, wherein the estimated present maximum voluntary contraction is based on a predetermined maximum voluntary contraction of the muscle of the user and on an estimated fatigue level of the muscle of the user.

17. The powered lower-body exoskeleton of claim 1 further comprising:
a suspension mechanism configured to be worn over shoulders of the user, the suspension mechanism configured to couple to the first upper support structure.

18. The powered lower-body exoskeleton of claim 1 wherein the first upper support structure is configured to be coupled to a pelvic region, and comprises a waist belt.
